(19) United States
(12) Patent Application Publication
(10) Pub. No.: US 2015/0094585 A1
(43) Pub. Date: Apr. 2, 2015

(54) ULTRASOUND TRANSDUCER WITH POSITION MEMORY FOR MEDICAL IMAGING

(71) Applicant: KONICA MINOLTA LABORATORY U.S.A., INC., San Mateo, CA (US)

(72) Inventor: Evgeny Ter-Ovanesyan, South San Francisco, CA (US)

(73) Assignee: KONICA MINOLTA LABORATORY U.S.A., INC., San Mateo, CA (US)

(21) Appl. No.: 14/502,900

(22) Filed: Sep. 30, 2014

Related U.S. Application Data
(60) Provisional application No. 61/884,808, filed on Sep. 30, 2013.

(51) Int. Cl. A61B 8/00

(52) U.S. Cl. CPC A61B 8/468; A61B 8/585
USPC 600/443; 600/459

(57) ABSTRACT

An ultrasound transducer device and control system disclosed here have the capability of storing information regarding the position of the transducer at which one ultrasound image is taken during an initial scan, and using the stored information to guide an operator to return the transducer to the same position during a subsequent scan. During the first scan, skin patterns are detected by a sensor, such as an optical sensor, capacitive sensor, or ultrasound sensor, and stored. During the subsequent scan, skin patterns are detected again, and compared to the stored skin pattern. A guidance message is generated based on the comparison to guide the user to return the ultrasound transducer device to the same position of the first scan.

### BACKGROUND OF THE INVENTION

1. Field of the Invention

This invention relates to an improved ultrasound transducer for medical imaging, and in particular, it relates to an ultrasound transducer capable of storing positions of the transducer relative to the patient's body during an imaging operation and recalling that position in a subsequent imaging operation.

2. Description of Related Art

Ultrasound imaging is one of the most widely used medical imaging modalities due to its safety, real-time capability and relatively low cost. However, interpretation of ultrasound images and finding a proper position and angle for an ultrasound transducer are not straightforward, and the corresponding training for its operator may take considerable time.

Devices aimed at helping the ultrasound operator with feedback information have been described. For example, U.S. Pat. No. 8382671 describes a handheld ultrasound probe with a force or torque sensor to provide feedback to the operator. U.S. Pat. Appl. Pub. No. 2013/0296707 describes an “ultrasound scanning system includes a graphical user interface that provides visually intuitive feedback to a user to assist the user in properly aligning an ultrasound scanner to a desired acquisition state” (see Abstract.) The devices includes a camera that can image a fiducial marker or other features on the patient's skin surface (see FIGS. 7 and 8, paras. [0096] to [0113]). U.S. Pat. Appl. Pub. No. 2014/0114193 describes a similar camera on the ultrasound system.

### SUMMARY

Embodiments of the present invention provides apparatus and related methods to store information regarding the position of an ultrasound transducer at which at least one ultrasound image is taken during an initial scan, and to use the stored information to guide an operator to return the ultrasound transducer to the same position during a subsequent scan. This may be generally referred to as “position memory” for the ultrasound transducer. Here, “position” refers to the position of the ultrasound transducer on the patient's body; since the transducer surface that contacts the body typically has an elongated shape, position refers to both the location and the orientation of the transducer on the skin surface.

An object of the present invention is to provide an ultrasound transducer device and related control apparatus that has position memory and can guide a user to a previous position on the patient's body.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve these and/or other objects, as embodied and broadly described, the present invention provides an ultrasound transducer device which includes: a housing having a detecting surface, the detecting surface including a first window and at least one second window; an ultrasound transducer disposed in the housing for generating and receiving ultrasound signals through the first window; and a sensor disposed in the housing for generating an image of skin of a patient when the skin is in contact with the second window of the detecting surface. The sensor may be an optical sensor, a capacitive sensor, or an ultrasound sensor.

In another aspect, the present invention provides a system for generating medical ultrasound images, which includes: a handheld ultrasound transducer device having a detecting surface, an ultrasound transducer for generating and receiving ultrasound signals through the detecting surface and a skin pattern detector for detecting a skin pattern of skin of a patient when the skin is in contact with the detecting surface; and a control device coupled to the handheld ultrasound transducer device, the control device being programmed to perform the following process: (a) analyzing and storing a first skin pattern generated by the handheld ultrasound transducer device; (b) comparing a second skin pattern generated by the handheld ultrasound transducer device with the stored first skin pattern; and (c) communicating a result of the comparing step (b) to a user.

In some embodiments, the skin pattern detector is an optical sensor, a capacitive sensor, or an ultrasound sensor, which is distinct from the ultrasound transducer. In other embodiments, the skin pattern detector is an ultrasound detector which shares a 2D array with the ultrasound transducer. In other embodiments, the ultrasound transducer is a Capacitance Micromachined Ultrasound Transducer (CMUT) device having a capacitor array, and the skin pattern detector shares the capacitor array with the ultrasound transducer.

In another aspect, the present invention provides a method for medical ultrasound imaging using a handheld ultrasound transducer device and a control device connected thereto, the handheld ultrasound transducer device having a detecting surface, an ultrasound transducer for generating and receiving ultrasound signals through the detecting surface and a skin pattern detector for detecting a skin pattern of skin of a patient when the skin is in contact with the detecting surface, the method including: during a first scan: (a) an operator placing the handheld ultrasound transducer device at a first position on a patient's body; (b) the ultrasound transducer device generating a first skin pattern of an area of the patient's skin, including an area of the skin which is in contact with a detecting surface of the ultrasound transducer device when the ultrasound transducer device is placed at the first position on the patient's body; (c) storing the first skin pattern; (d) generating ultrasound scan using the handheld ultrasound transducer device at the first position; and during a second scan: (e) an operator placing the handheld ultrasound transducer device at a second position on the patient's body; (f) the ultrasound transducer device generating a second skin pattern of an area of the patient's skin which is in contact with the detecting surface of the ultrasound transducer device when the ultrasound transducer device is placed at the second position on the patient's body; (g) the control device comparing the second skin pattern with the stored first skin pattern; (h) the control device generating a message based on the comparison of step (g); (i) repeating steps (f) to (h) after the operator moves the handheld ultrasound transducer device to another second position on the patient's body; (j) generating ultrasound scan using the handheld ultrasound transducer device at the second position.

In another aspect, the present invention provides a computer program product comprising a computer usable non-transitory medium (e.g. memory or storage device) having a computer readable program code embedded therein for controlling a data processing apparatus, the computer readable program code configured to cause the data processing apparatus to execute the above method.

It is to be understood that both the foregoing general description and the following detailed description are exemplary and explanatory and are intended to provide further explanation of the invention as claimed.

### BRIEF DESCRIPTION OF THE DRAWINGS

FIG. 1 illustrates an ultrasound transducer device according to a first embodiment of the present invention. Ultrasound transducer device according to a second and third embodiment of the present invention generally have a similar appearance.

FIG. 2 schematically illustrates am ultrasound imaging method according to an embodiment of the present invention.

FIG. 3 schematically illustrates an ultrasound imaging system according to embodiments of the present invention.

### DETAILED DESCRIPTION OF PREFERRED EMBODIMENTS

An ultrasound transducer according to embodiments of the present invention has the capability of storing information regarding the position of the transducer at which one ultrasound image is taken during an initial scan, and using the stored information to guide an operator to return the transducer to the same position during a subsequent scan.

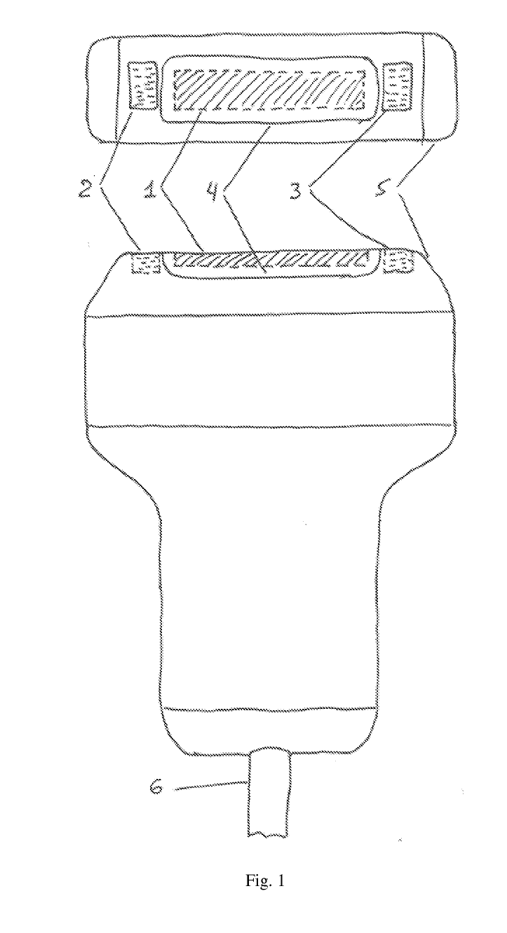

FIG. 1 shows a handheld ultrasound transducer device according to a first embodiment of the present invention. A top view is shown in the upper part of FIG. 1, and a side view is shown in the lower part of FIG. 1. An ultrasound transducer 1 is used for generation and reception of ultrasound waves. Transducer 1 is mounted inside an ultrasound transducer holder (housing) 5; a part of it is exposed on the top surface of the holder and may be covered with a protective layer 4. The size of the imaging area of the transducer 1 on the surface of the holder 5 is approximately 2 by ½ inches for a typical device, but other sizes can be used as well. The parts of the ultrasound transducer device and related control apparatus that perform the ultrasound imaging function are well known in the art and a detailed description is omitted here. Cord 6 transmits electrical signals between the handheld ultrasound transducer device and the control apparatus.

One or more sensors are provided in the ultrasound transducer holder 5, with windows on the top surface of the holder 5 next to the window of the ultrasound transducer 1. FIG. 1 shows an embodiment with two sensors (windows) 2 and 3 which are located on the opposite sides of the transducer 1; however, other numbers and locations of the sensor may be used as well. During imaging, the top surface of the holder 5, including the exposed transducer area 1 and the windows of the sensor, is in contact with the patient's skin. In other words, the window of the sensor and the window of the ultrasound transducer can simultaneously contact the patient's skin. In the first embodiment, the sensors 2 and 3 are optical sensors which can detect skin surface features such as ridges and valleys. For example, optical sensors similar to the sensors used for fingerprint scanning may be used here. One example is an optical sensor device described in U.S. Pat. Appl. Pub. 2012/0287254. The optical sensors 2 and 3 may be based on optical Frustrated Total Internal Reflection, as commonly used in fingerprint scanning. This technique uses a prism, or one or more microprism layers to reduce dimensions of the optical part of the device. Each sensor 2, 3 includes an imaging unit (including e.g. a light source, a detector, and optical components, not shown in FIG. 1) disposed inside the holder 5, for sensing the skin pattern present at the corresponding sensor window on the top surface of the transducer holder 5.

The task of scanning human skin to find specific skin patterns is technically similar to the task of fingerprint scanning used for biometric applications. Fingerprint recognition is a well developed field. Two papers are cited here to show the general state of the art:

Fingerprint Recognition, by Fernando Alonso-Fernandez and Josef Bigun, Julian, Fierrez, Hartwig Fronthaler, Klaus Kollreider, Javier Ortega-Garcia, in Guide to Biometric Reference Systems and Performance Evaluation (2009), Chap. 4, available on the Internet at http://www2.hh.se/staff/josef/public/publications/alonso-fernandez09chapter.pdf. This paper describes acquiring fingerprint images using optical sensors, and fingerprint recognition algorithms.

Biometric Security Using Finger Print Recognition, by Subhra Mazumdar, Venkata Dhulipala, University of California, San Diego, available on the Internet at http://cseweb.ucsd.edu/classes/fa08/cse237a/finalproject/smazumdar_final_report.pdf. This paper describes acquiring fingerprint images using capacitive sensors, and fingerprint recognition algorithms.

In the first embodiment of the present invention, a skin pattern recognition algorithm similar to known fingerprint recognition algorithms may be used. For example, the algorithm may be based on recording a microscopic skin pattern containing ridges and classifying the pattern based on so called minutia points. The minutia points are typically places where ridges end and/or bifurcate (split). After identifying the minutia points, distances between them may be calculated. Such skin pattern features can be stored, and compared with skin pattern features detected during a subsequent scan.

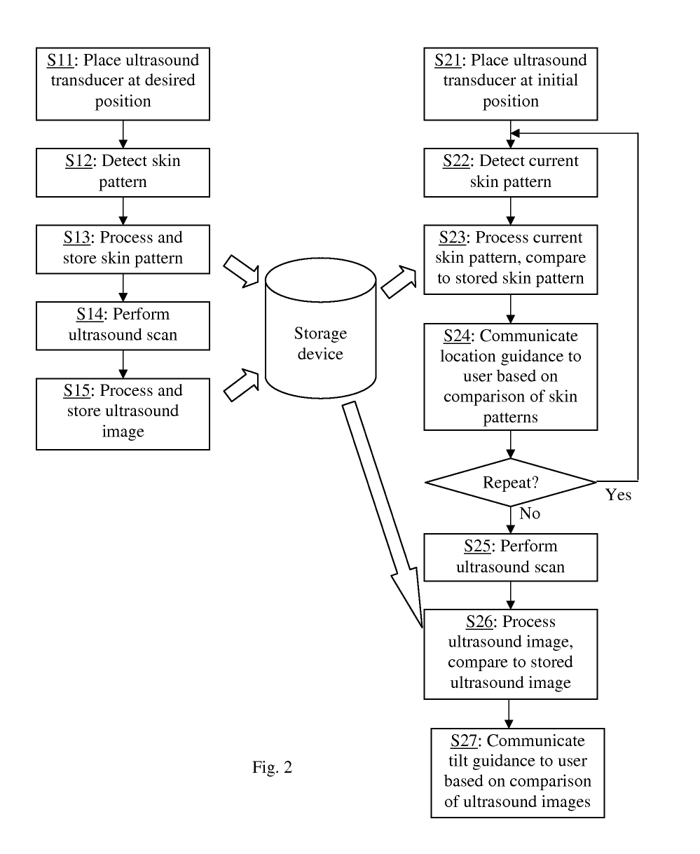

In operation (refer to FIG. 2), during a first ultrasound scan (steps S11 to S15), after the ultrasound transducer device is placed in a proper position (step S11), the operator activates a control switch to make an optical image of the skin area with sensors 2 and 3 (step S12), and carries out an ultrasound scan at that position (step S14). The skin pattern is analyzed (e.g. the minutia points are detected) and stored (step S13). For convenience, the transducer position during the first scan is referred to as the target position and the corresponding skin pattern is referred to as the target skin pattern.

In one implementation, during the first scan, multiple skin patterns at different locations are imaged with the optical sensors while the operator moves the transducer device across the patient's body approaching a desired position. It is preferable to image a sufficiently large area of the patient's body surrounding the desired position. Preferably, the imaged skin areas of successive images overlap with each other, so that the images can be stitched together to form an image of a larger area of the skin. The desired position may be marked on this larger image. This will make it easier to return the transducer device to the desired position in subsequent scans.

During a subsequent scan (steps S21 to S27), the ultrasound transducer device is placed at an initial position (step S21), and the optical sensors 2 and 3 are activated to detect the skin patterns at the initial position (referred to as the current skin pattern for convenience) (step S22). The initial transducer position for the subsequent scan should be sufficiently close to the target position so that the initially detected skin patterns overlaps at least to some extend the stored skin pattern at the target position. If during the first ultrasound scan a larger image of the skin pattern was formed by stitching multiple images together, then the initial transducer position for the subsequent scan only needs to overlap this larger area. Then, the current skin pattern data is processed, and a measure that indicates the closeness of the current skin pattern and the target skin pattern is calculated (step S23). The measure of closeness may be, for example, a correlation between the two patterns, or a translation distance and rotation angle between the two positions, etc. In particular, if a larger image of the skin area was stored during the first scan, it is possible to find the current position on the larger image, and calculate a translation distance and a rotation angle relative to the current position that would place the transducer at the target position.

Then, the operator moves the transducer by a desired amount, and the sensor detects the updated skin pattern (step S22). The measure of closeness between the updated skin pattern and the target skin pattern is calculated (step S23). These steps are repeated as desired. During this process, the calculated measure of closeness is communicated to the operator by suitable messages to guide the operator to move the ultrasound transducer toward the target position (step S24). The message may be a visual display or an audible message, or other suitable forms of communication. For example, if the measure of closeness is a correlation value (which indicates how similar the two patterns are but does not indicate a direction of improvement), the message may be a visual display such as a bar of a variable length displayed on a screen of the control apparatus or a light with variable intensity, or an audible message such as a series of beeps of a variable frequency or volume (e.g. more frequent beeps indicates the position is getting closer, with continuous tone indicating the target location has been reached); etc. If the measure of closeness gives the translation distance and rotation angle that the transducer should be moved toward the target position, the message can direct the operator to move (including rotate) the transducer in a particular was to approach the target position.

Using the messages as guidance, the operator moves the transducer gradually across the skin to reach the target position, where ultrasound scans may be taken (step S25) for “before and after” comparison or other purposes. Based on practical experience, it is estimated that the precision goal for transducer placement is approximately 3 mm; i.e., if the transducer can be returned to a position within 3 mm from the target position, the second scan will be satisfactory for the goal of operator training, scan image comparison, etc.

An ultrasound transducer device according to a second embodiment of the present invention has an exterior structure generally similar to that shown in FIG. 1, but the sensors (e.g. sensors 2 and 3) are capacitive sensors. Capacitive sensors can detect skin patterns based on the fact that the skin is a conductor and can be considered as a plate of a capacitor. Each capacitive sensor includes an array of capacitors which detect a difference in capacitance between ridges and valleys in the skin. Capacitive sensors have been used in fingerprint scanning (see, e.g. Subhra Mazumdar et al., Biometric Security Using Finger Print Recognition, cited earlier); they may be adapted for use in the ultrasound transducer of the second embodiment. The skin patterns detected by the capacitive sensors are used to guide the operator to return the ultrasound transducer to a target position based on previously stored skin patterns. Similar skin pattern comparison algorithm and guidance method as in the first embodiment may be applied here. The use of the ultrasound transducer device for returning to a previous position is similar to that of the first embodiment.

An ultrasound transducer device according to a third embodiment of the present invention has an exterior structure generally similar to that shown in FIG. 1, but the sensors (e.g. sensors 2 and 3) are ultrasound sensors. For example, the sensors may be 2D ultrasound arrays capable of high-resolution imaging of the skin to detect skin texture. A 3D ultrasound image is generated by the sensors 2 and 3, and data regarding the outer sub-mm layer of the tissue is used to construct a 2D surface map of the skin, which includes skin patterns such as ridges and valleys. Similar skin pattern comparison algorithm and guidance method as in the first embodiment may be applied here. The use of the ultrasound transducer device for returning to a previous position is similar to that of the first embodiment.

In a fourth embodiment of the present invention, skin pattern is detected by ultrasound imaging, but unlike the third embodiment, no separate ultrasound sensors 2, 3 are required; rather, the same ultrasound transducer 1 used for generating the medical image is used to detect skin patterns. The ultrasound transducer 1 is preferably a 2D array for 3D ultrasound imaging; it is operated in a regular ultrasound imaging mode to generate 3D ultrasound image, and data regarding the outer sub-mm layer of the tissue is used to construct a 2D surface map of the skin. A skin pattern generated this way is used for guidance. The hardware of the ultrasound transducer device of the fourth embodiment is similar to a conventional ultrasound transducer device, but control and data processing software is provided to perform skin pattern detection and analysis and to generate guidance messages. Similar skin pattern comparison algorithm and guidance method as in the first embodiment may be applied here. The use of the ultrasound transducer device for returning to a previous position is similar to that of the first embodiment.

In a fifth embodiment of the present invention, the ultrasound transducer device is based on the well-known CMUT (Capacitance Micromachined Ultrasound Transducer) technology, where ultrasound is generated and received by an array of capacitors with a moving membrane as one of the capacitor plates. As CMUT technology is well known, a detailed description of this technology is omitted here. A general description of CMUT can be found in Khuri-Yakub Ultrasonic Group, General Description and Advantages of CMUTs, available on the Internet at http://www-kyg.stanford.edu/khuriyakub/opencms/en/research/cmuts/general/index.html.

Using the capacitor array of a CMUT device for fingerprint detection has been suggested; see Pavlo Fesenko, Capacitive Micromachined Ultrasonic Transducer (CMUT) For Biometric Applications, Thesis for the Degree of Erasmus Mundus Master of Nanoscience and Nanotechnology, Department of Microtechnology and Nanoscience, Chalmers University Of Technology, Goteborg, Sweden, 2012, available on the Internet at http://publications.lib.chalmers.se/records/fulltext/166084.pdf. This thesis suggests using CMUT/capacitance combination for improved fingerprint detection. As ultrasound works better than capacitance in case of contaminated fingers, the thesis suggests using both an ultrasound mode and a capacitance mode of the CMUT device for fingerprint detection. Note that in this thesis, the CMUT transducer is not used for medical imaging, but only for scanning fingerprints.

According to the fifth embodiment, the array of capacitors in a CMUT device is used as capacitive sensors to detect skin patterns. The transducer device is controlled to operate in two modes: The first mode is the ultrasound mode, which operates the same as in conventional CMUT devices to generate ultrasound images. The second mode is a capacitance mode, in which the array of capacitors is used to detect skin patterns using capacitive sensing. Thus, skin pattern is acquired in the capacitance mode, and the ultrasound mode is used for regular ultrasound imaging. In this embodiment, no additional sensor is required, beyond the capacitors of the existing CMUT devices, to detect skin patterns. A control section is provided to control the capacitor arrays to perform capacitive sensing, and a switch is provided to switch between the ultrasound mode and the capacitance mode. The hardware of the ultrasound transducer device of the firth embodiment is otherwise similar to that of a conventional CMUT device. Control and data processing software is provided. Similar skin pattern comparison algorithm and guidance method as in the first embodiment may be applied here. The use of the ultrasound transducer device for returning to a previous position is similar to that of the first embodiment. The fifth embodiment is the most preferred embodiment of the present invention.

In the fourth and fifth embodiment, the area of the detector used for skin pattern detection is approximately the same as the area of the ultrasound transducer 1. The size of the transducer, and hence the area of skin pattern detection, is approximately 2 by ½ inches for a typical device. Larger areas for skin pattern detection is desirable because, as mentioned earlier, during a subsequent scan, the initial position of the transducer should have some overlap with the skin area detected in the previous scan.

In addition to position, another important parameter for ultrasound transducer placement is the tilt angle of the device. In the orientation of FIG. 1, side view, the tilt angle refers to the angle of a tilt (or rocking motion) of the transducer handle in and out of the plane of the drawing sheet. Because the skin and the tissue under the skin are typically soft, the transducer can be tilted to a certain degree while maintaining contact with the skin, by pressing the skin and the tissue down. This tilt angle changes the angle of the transducer relative to the organ being imaged, and typically leads to different ultrasound images at the same location. The change in the tilt angle will not change the skin pattern being detected by the sensors, so it is difficult to provide guidance for a proper tilt angle by using skin patterns. During a subsequent scan, to help returning the transducer to the tilt angle of a previous scan, a current ultrasound image may be compared with a stored ultrasound image from the previous scan to indicate how close the tilt angles are. In one example, the ultrasound image from the previous scan is low-pass filtered and converted to binary to reduce amount of information, and then stored. Then, during the subsequent scan, a current ultrasound image is taken, and processed by the same procedure (low pass filtering and converting to binary image), and a cross-correlation between the stored and current ultrasound images is calculated. This cross-correlation value may be used to guide the operator to return the transducer to the tilt angle of the first scan. In addition, comparison of the ultrasound images (e.g. the cross-correlation value) may be used to verify that the position of the transducer is close to the position of the previous scan. These steps are shown as steps S15 and S26 to S27 in FIG. 1.

In addition, the skin pattern detected by the sensors 2, 3 may be used as an indicator of force applied by the ultrasound transducer housing 5 (the top surface of it) to the patient's body. When the force increases, the skin will tend to expand, and the corresponding skin pattern will be slightly distorted but will still have the same skin features. For example, the minutia points in the skin pattern can still be recognized, but distance between them will tend to be increased, typically proportionally, if a larger force is applied. Thus, by comparing the detected skin patterns of the subsequent scan and the first scan, the system may generate an indication (e.g., via visual display or audible message) of the applied force as feedback to the operator. This feedback can be in addition to the feedback regarding the position of the transducer. The feedback regarding the applied force can be used to guide the operator until the desired amount of force is applied, so that the condition of the previous including the force scan can be reproduced.

The system described herein can additionally be used to provide evaluation of the operator's skills, by analyzing the time it takes for an operator to find the target location. It can be done simply by recording the time it takes, or by matching skin patterns and evaluating how the operator moves a transducer toward the target location. This evaluation may result in a score which will be displayed to the operator.

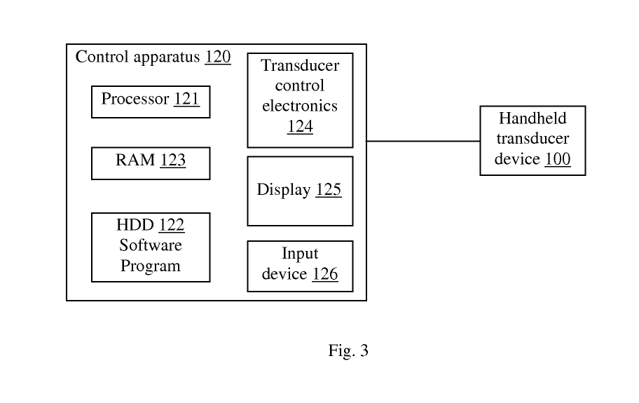

An ultrasound imaging system according to embodiments of the present invention is schematically shown in FIG. 3. The system includes a handheld ultrasound transducer device 100 and a control apparatus 120. The structure of the handheld ultrasound transducer device 100 is described in detail above. The control apparatus 120 includes a processor 121, a storage device (e.g. hard disk drive) 122, and an internal memory (e.g. a RAM) 123. The storage device 122 stores software programs, which are read out to the RAM 123 and executed by the processor 121 to carry out the various control and data processing functions described above. The control apparatus further includes an electronics module 124 for transducer control, a display 125, and an operator input device 126, as well as other suitable components such as a sound producer, not shown in FIG. 3. The visual and audible guidance messages described earlier are preferably produces by the control apparatus, but they can also be produced by the handheld device, in which case suitable hardware is provided in the handheld device.

The technology disclosed here can be helpful in many situations. For example, it is helpful in case when an experienced sonographer performs an ultrasound scan and less experienced sonographers try to repeat it for training purposes. It is also helpful to be able to get back exactly to the same position to compare “before” and “after” ultrasound images of an organ after administering a drug, performing exercise, etc. Furthermore, as the price of ultrasound transducers comes down and the computing power of smart phones, tablets, and personal computers goes up, ultrasound imaging may become more available for the general population. Therefore, there will be a need to teach inexperienced users to use ultrasound equipment. In such cases, it will be helpful to have an ultrasound transducer with position memory. One example of its use is monitoring a fetus during pregnancy. After doctor shows a patient how to image a fetus for the first time, position of the ultrasound transducer device can be stored in memory and the device will provide guidance to the patient on proper positioning of the device and getting a good image at home.

It will be apparent to those skilled in the art that various modification and variations can be made in the ultrasound transducer device and system and related method of the present invention without departing from the spirit or scope of the invention. Thus, it is intended that the present invention cover modifications and variations that come within the scope of the appended claims and their equivalents.

What is claimed is:

1. An ultrasound transducer device comprising:
a housing having a detecting surface, the detecting surface including a first window and at least one second window;
an ultrasound transducer disposed in the housing for generating and receiving ultrasound signals through the first window; and
a sensor disposed in the housing for generating an image of skin of a patient when the skin is in contact with the second window of the detecting surface.

2. The ultrasound transducer device of claim 1, wherein the sensor is an optical sensor, a capacitive sensor, or an ultrasound sensor.

3. The ultrasound transducer device of claim 1, wherein the sensor is an ultrasound sensor which shares a 2D array with the ultrasound transducer and the first window and the second window are the same windows.

4. The ultrasound transducer device of claim 1, wherein the first window and the second window are different windows.

5. The ultrasound transducer device of claim 4, wherein the first window and the second window are disposed to come in contact with the skin of the patient simultaneously.

6. A system for generating medical ultrasound images, comprising:
a handheld ultrasound transducer device having a detecting surface, an ultrasound transducer for generating and receiving ultrasound signals through the detecting surface and a skin pattern detector for detecting a skin pattern of skin of a patient when the skin is in contact with the detecting surface; and
a control device coupled to the handheld ultrasound transducer device, the control device being programmed to perform the following process:
(a) analyzing and storing a first skin pattern generated by the handheld ultrasound transducer device;
(b) comparing a second skin pattern generated by the handheld ultrasound transducer device with the stored first skin pattern; and
(c) communicating a result of the comparing step (b) to a user.

7. The system of claim 6, wherein the skin pattern detector is an optical sensor, a capacitive sensor, or an ultrasound sensor, and is distinct from the ultrasound transducer.

8. The system of claim 6, wherein the skin pattern detector is an ultrasound detector which shares a 2D array with the ultrasound transducer.

9. The system of claim 8, wherein the ultrasound transducer is a Capacitance Micromachined Ultrasound Transducer (CMUT) device having a capacitor array, and wherein the skin pattern detector shares the capacitor array with the ultrasound transducer.

10. A method for medical ultrasound imaging using a handheld ultrasound transducer device and a control device connected thereto, the handheld ultrasound transducer device having a detecting surface, an ultrasound transducer for generating and receiving ultrasound signals through the detecting surface and a skin pattern detector for detecting a skin pattern of skin of a patient when the skin is in contact with the detecting surface, the method comprising:
during a first scan:
(a) an operator placing the handheld ultrasound transducer device at a first position on a patient's body;
(b) the ultrasound transducer device generating a first skin pattern of an area of the patient's skin, including an area of the skin which is in contact with a detecting surface of the ultrasound transducer device when the ultrasound transducer device is placed at the first position on the patient's body;
(c) storing the first skin pattern;
(d) generating ultrasound scan using the handheld ultrasound transducer device at the first position; and
during a second scan:
(e) an operator placing the handheld ultrasound transducer device at a second position on the patient's body;
(f) the ultrasound transducer device generating a second skin pattern of an area of the patient's skin which is in contact with the detecting surface of the ultrasound transducer device when the ultrasound transducer device is placed at the second position on the patient's body;
(g) the control device comparing the second skin pattern with the stored first skin pattern;
(h) the control device generating a message based on the comparison of step (g);
(i) repeating steps (f) to (h) after the operator moves the handheld ultrasound transducer device to another second position on the patient's body;
(j) generating ultrasound scan using the handheld ultrasound transducer device at the second position.
